(19) United States
(12) Patent Application Publication
(10) Pub. No.: US 2021/0100568 A1
(43) Pub. Date: Apr. 8, 2021

(54) CUTTING AND DRILLING TEMPLATE FOR UNICONDYLAR KNEE ARTHROPLASTY

(71) Applicant: Christiaan Rudolf OOSTHUIZEN, Johannesburg (ZA)

(72) Inventor: Christiaan Rudolf OOSTHUIZEN, Johannesburg (ZA)

(21) Appl. No.: 17/050,335

(22) PCT Filed: Apr. 26, 2019
(86) PCT No.: PCT/IB2019/053440

§ 371 Date: Oct. 23, 2020

(30) Foreign Application Priority Data
Apr. 26, 2018 (ZA) 2018/02779

(51) Int. Cl. A61B 17/17

(52) U.S. Cl. CPC A61B 17/1764; A61B 17/155

(57) ABSTRACT

This invention relates to a positioning template used to determine correct cutting planes and angles and drilling positions during surgical procedures, and more particularly, but not exclusively, to a femoral cutting template (10) used in the preparation of a femoral condyle during a unicondylar knee arthroplasty (UKA). The femoral cutting template (10) comprises attachment means (12) for releasably attaching the template to a tibiofemoral spacer shim (50), provided between a proximal tibia and a femoral condyle. The template further comprises a main body (14) defining first and second guide apertures (16,18) for guiding first and second tools respectively. The first and second tools are used for creating first and second receiving formations (206,208), in the femoral condyle, which are created for receiving first and second parts of a femoral component forming part of an unicondylar knee prosthesis.

### INTRODUCTION AND BACKGROUND

This invention relates to a positioning template used to determine correct cutting planes and angles and drilling positions during surgical procedures, and more particularly, but not exclusively, to a femoral cutting template for use in a unicondylar knee arthroplasty (UKA) procedure.

UKA, also referred to as unicompartmental knee arthroplasty or partial knee replacement, is a procedure wherein only part of a knee of a patient is replaced or resurfaced to alleviate severe discomfort caused by arthritic degeneration of either the lateral or medial condylar cartilage of the knee. It is indicated as a less invasive alternative to total knee arthroplasty (TKA), wherein the condyles of both the distal femur and proximal tibial surfaces are completely resected and resurfaced by femoral and tibial prosthetic devices, commonly referred to as femoral- and tibial components respectively.

The advantages of the UKA over TKA are well documented UKAs find specific application for patients that only experience arthritis in a single compartment of the knee, instead of in both, with the associated retention of major knee ligaments coupled with significantly decreased post-operative pain, discomfort and rehabilitation requirements being further associated advantages leading to the increase in popularity of this procedure with orthopaedic surgeons, patients and medical insurance providers.

One of the advantages of UKAs over TKAs, and specifically in cases where stability providing structures (ligaments and tendons) are retained, is the retention of the patient's constitutional alignment. Constitutional alignment refers to the natural anatomical alignment of a specific patient. In cases where a prosthesis replaces the function of the stability providing structures, constitutional alignment is often lost.

Due to the retention of major ligaments, such as the anterior- and posterior cruciate ligaments, medial and lateral collateral ligaments and the patellar tendon, that is achieved by resurfacing of only part of the knee in a UKA procedure, instead of sacrificing these ligaments when performing a TKA procedure, it is imperative that the femoral and tibial surfaces are resected accurately to achieve correct post-operative alignment of the knee joint, and to retain constitutional alignment of the knee. Because of the less invasive nature of a UKA (compared to a TKA), the decreased post-operative recovery time and the degree to which constitutional alignment and normal knee function can be retained, a UKA should always be preferred over a TKA (in suitable cases).

Because of a UKA's reliance on the patient's stability providing structures, the accuracy of the placement of the prosthetic components of the UKA is crucial in the success of the arthroplasty, the post-operative recovery of the patient, and the degree to which normal function of the joint is obtained post-operatively. Currently available prosthetic devices and cutting templates utilised during knee surgery place a significant reliance on subjective factors, which are informed by the experience and expertise of the operating surgeon. For this reason, the reliability and repeatability of these procedures cannot be guaranteed.

The success of a UKA still heavily relies on the experience and expertise of the surgeon, who often has to adjust resections based on patient-specific anatomy, to ensure that the prosthetic devices fit properly so as to ensure accurate post-operative joint alignment. There is currently no standardised instrumentation or femoral and tibial cutting template or guide that a surgeon can rely on to provide adequate resected femoral and tibial plateaus for insertion of femoral and tibial components. Because of this, a reluctance remains on the part of surgeons to perform UKAs, and on the part of patients to opt for a UKA over a TKA, even in cases where a UKA is indicated or may be suitable.

A generally utilised cutting and drilling template or guide for performing mobile UKA procedures operates as follows:
preparation of the knee joint is done by making necessary incisions into the knee, with excisions of fat and other tissue so as to expose the anterior tibia and distal femur, and removal of osteophytes;
a tibial cutting template or guide is positioned relative to the medial- or lateral condyle, as the case may be, and is fixed in position at the distal end of the template or guide by a strap fitted around the leg of the patient slightly superior of the ankle joint, and by securing it to the tibia at the proximal end by a surgical pin or screw once the surgeon is satisfied with the alignment of the tibial cutting template or guide relative to the tibia and the affected condyle, albeit medial or lateral. Once in position, the tibial surface is resected with a surgical saw that is guided by the superior surface of the tibial cutting template or guide;
the surgeon then tests the space that was opened by the tibial resection to determine if a tibial component and a spacer bearing of at least 7 mm in thickness will fit into the excised space and engage with the femoral condyle. If the space is insufficient, the tibial cutting template or guide has to be removed, repositioned and the tibia cut further. Alternatively, a ruler is used to determine placement of the tibial cutting template or guide by measuring the space between the femoral condyle and the tibial plateau that will be required for a tibial component and a spacer bearing, and then setting the tibial cutting template or guide in place. However, the process of resecting the tibial plateau by fitting the tibial cutting template or guide by either spacing it relative to structures on the anterior tibia or by measuring a distance from the femoral condyle indicates the less than optimal reliance on the level of expertise of the surgeon, instead of having an objectively repeatable device and process available wherein less reliance is placed on subjective factors such as surgeon expertise;
once the surgeon is satisfied that a suitable tibial resection has been achieved, the tibial cutting template or guide is removed;
a hole is drilled in the associated femoral condyle, and an intramedullary rod is inserted through the hole and into the femoral medulla;
a femoral template or guide is positioned on the femoral condyle by a connector connected to the end of the intramedullary rod at the point where it is exposed from the femoral condyle, and once the surgeon is satisfied of the position of the femoral cutting template, the template or guide is attached to the femoral condyle by a surgical pin or screw, whereafter the intramedullary rod is removed, and the posterior condyle is resected. The femoral cutting template also indicates where holes in the femoral condyle have to be drilled for attaching the femoral component to the resected femur, and if the initial placement of the femoral component as indicated by the connector is incorrect, the positioning of holes and resection of the femoral condyle will be negatively affected. Currently available femoral templates or guides therefore rely almost exclusively on surgeon expertise for correct initial placement of the femoral cutting template relative to the femoral condyle;
once the posterior femoral condyle has been resected and a guiding hole for the positioning of the rotary mill and later attachment of the femoral component has been drilled, the femoral cutting template is removed;
a spigot is inserted into a guiding hole for the rotary mill, and the condylar surface is milled away by a rotary mill, with excess bone being cleared from the milled surface by a chisel;
once the femoral and tibial condyles have been sufficiently resected, the femoral and tibial components are inserted and cemented in place, if the surgeon is satisfied with the fit of the components and their interaction with each other. A polymeric spacer bearing is inserted between the tibial and femoral components.

Whilst other methods and cutting templates or guides are available, the above description pertains to the process and guide that is preferred and most often used in the industry at present.

### OBJECT OF THE INVENTION

It is accordingly an object of the current invention to provide a cutting template or guide for femoral resections during UKA procedures that provides for reliably repeatable and accurate placement of femoral components in situ, and which decreases the reliance on surgical expertise and other subjective factors when performing UKA procedures.

### SUMMARY OF THE INVENTION

According to a first aspect of the invention there is provided a femoral cutting template comprising:
attachment means for releasably attaching the template to a tibiofemoral spacer shim which is operatively provided between a proximal tibia and a femoral condyle;
a main body defining a first guide aperture for guiding a first tool, used for creating a first receiving formation in the femoral condyle, the first receiving formation for receiving a first part of a femoral component; and
a second guide aperture defined within the main body, for in use, guiding a second tool used to create a second receiving formation in the femoral condyle, the second receiving formation for receiving a second part of the femoral component, the second guide aperture spaced a first predetermined distance from the first guide aperture, and a second predetermined distance from the attachment means.

The first guide aperture may typically comprise a first guide hole, in which case the first tool may comprise a first drill bit so that the first receiving formation may be a first hole created in the femoral condyle by the first drill bit. Consequently, the first part of the femoral component that is received in the first hole may comprise a first peg located on an inner aspect of the femoral component.

The second guide aperture may comprise a guide slot, while the second tool may comprise a cutting blade. The second receiving formation may therefore be a portion of the condyle resected by the cutting blade, so that the second part of the femoral component may comprise a flat surface on an inner aspect of the femoral component.

The main body may define a third guide aperture which may take the form of a second guide hole, for guiding a second drill bit. The drill bit may be used for creating a second hole in the femoral condyle, which second hole may be for receiving a second peg located on the inner aspect of the femoral component.

The first and third guide apertures may be spaced a third predetermined distance from each other.

The main body may define a viewing aperture between the first and second guide apertures, which may operatively provide viewing access of an anterior aspect of the femoral condyle.

The main body may define a first securing formation, which may take the form of a first securing hole. The first securing hole may be arranged towards a first side portion of the main body, and the first securing hole may extend obliquely relative to the first guide aperture. The first side portion may be a left side portion of the main body. The first securing hole may be provided for releasably receiving a pin of a connector device which, in use, may be fixed to an intramedullary rod during a medial unicondylar arthroplasty. The first securing hole may extend substantially downwardly and outwardly from an anterior face of the main body, towards a posterior face of the main body.

The main body may furthermore define a second securing formation, which may take the form of a second securing hole. The second securing hole may be arranged towards a second side portion of the main body, and the second securing hole may extend obliquely relative to the first guide aperture. The second side portion may be a right side portion of the main body. The second securing hole may be provided for releasably receiving a pin of a connector device which, in use, may be releasably fixed to an intramedullary rod during a lateral unicondylar arthroplasty. The second securing hole may extend substantially downwardly and outwardly from an anterior face of the main body, towards a posterior face of the main body.

The attachment means may comprise first and second opposing hook-shaped formations which may project from the main body, and typically from a bottom portion of the main body. The first and second hook-shaped formations may constitute a pair defining a T-shaped slot therebetween. In use, the T-shaped slot may receive rim formations formed on the spacer shim, thereby releasably to attach the femoral cutting template to the spacer shim.

A posterior face of the of the main body, which is operatively arranged to face the femoral condyle, may be concave.

Typically, the main body may be of unitary construction and manufactured from a metal, such as titanium, aluminium or ferrous alloys such as stainless steel. The main body may be manufactured through a process of metallic machining or casting.

In an alternative embodiment of the first aspect of the invention, the second guide aperture may comprise a secondary guide hole, the second tool may comprise a secondary drill bit and the second formation may comprise a secondary peg located on an inner aspect of the femoral component.

According to a second aspect of the invention there is provided a tibiofemoral spacer shim comprising:
a first surface for operatively contacting a proximal tibia;
a second surface for operatively contacting a surface of a femoral condyle, the first and second surfaces separated by a spacing distance; and
an attachment formation for facilitating a femoral cutting template to be releasably attached thereto.

The attachment formation may comprise first and second, longitudinally extending lateral shoulder formations, which may have upper surfaces which may be substantially flush with the second surface. Consequently, the shim may be in the form of a substantially T-shaped elongate body.

The tibiofemoral spacer shim may further comprise a removable handle, which may in in use extend rearwardly from a rear portion of the elongate body. The first and second surfaces may typically extend substantially parallel to each other.

According to a third aspect of the invention there is provided a femoral cutting assembly, consisting of:
a tibiofemoral spacer shim, comprising first and second surfaces separated by a spacing distance, the first and second surfaces for operatively contacting a proximal tibia and a surface of a femoral condyle respectively; and
a femoral cutting template, releasably attached via an attachment means to the tibiofemoral spacer shim, the femoral cutting template comprising:
   a main body defining a first guide aperture for guiding a first tool, used for creating a first receiving formation in the femoral condyle, the first receiving formation for receiving a first part of a femoral component; and
   a second guide aperture defined within the main body for guiding a second tool used to create a second receiving formation in the femoral condyle, the second receiving formation for receiving a second part of the femoral component, the second guide aperture spaced a first predetermined distance from the first guide aperture, and a second predetermined distance from the attachment means.

The femoral cutting assembly may therefore consist of a tibiofemoral spacer shim according to the second aspect of the invention, and a femoral cutting template according to the first aspect of the invention, which may be releasably attached to the tibiofemoral spacer shim.

According to a fourth aspect of the invention there is provided a method of preparing a femoral condyle for the placement of a femoral component forming part of a unicondylar knee prosthesis, the method comprising the steps of:
placing a tibiofemoral spacing shim between a proximal tibia and a surface of the femoral condyle;
releasably attaching a femoral cutting template via an attaching means to the tibiofemoral spacing shim, the femoral cutting template comprising first and second guide apertures;
providing a first tool into the first guide aperture and utilising the first tool to create a first receiving formation in the femoral condyle; and
providing a second tool into the second guide aperture and utilising the second tool to create a second receiving formation in the femoral condyle.

### BRIEF DESCRIPTION OF THE ACCOMPANYING DIAGRAMS

The invention will now be described further by way of non-limiting examples with reference to the accompanying drawings, wherein:

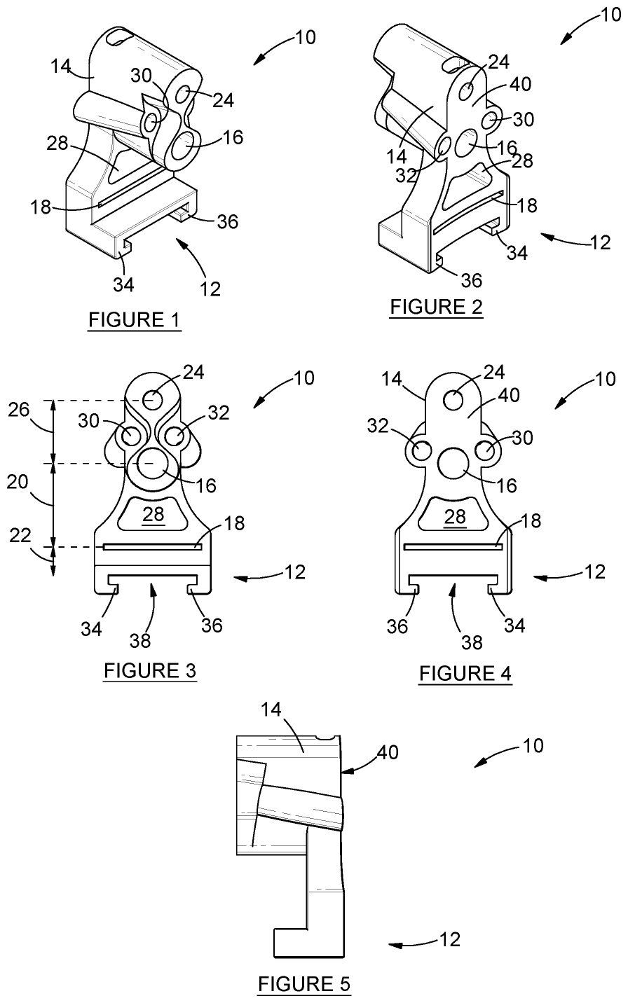

FIG. 1 is a front perspective view of a femoral cutting template according to one aspect of the invention;

FIG. 2 is a rear perspective view of the femoral cutting template of FIG. 1;

FIG. 3 is a front view of the femoral cutting template of FIG. 1;

FIG. 4 is a rear view of the femoral cutting template of FIG. 1;

FIG. 5 is a side view of the femoral cutting template of FIG. 1;

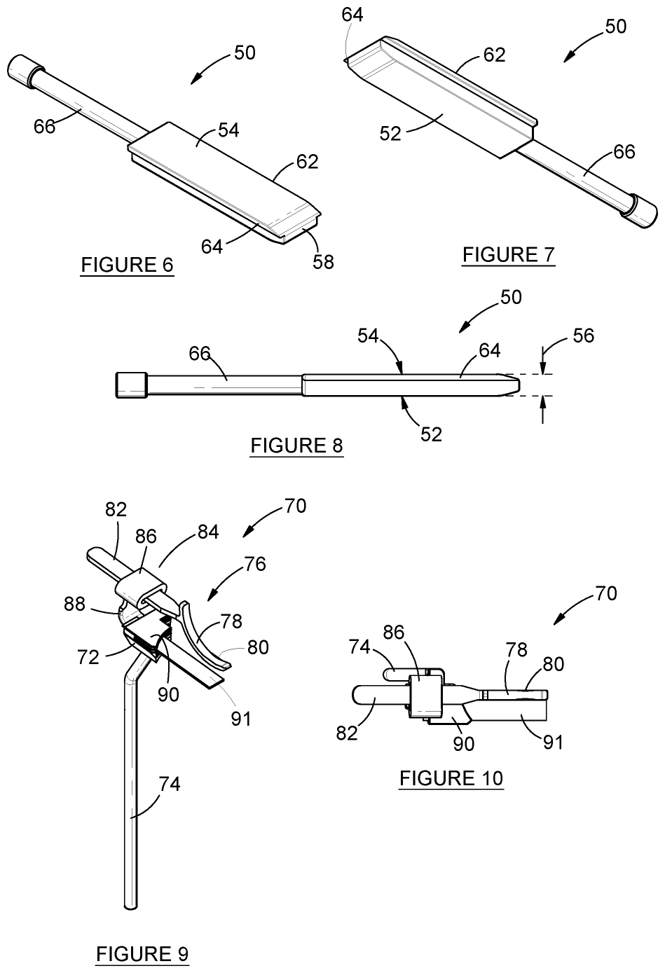

FIG. 6 is a top perspective view of a spacer shim according to the invention;

FIG. 7 is a bottom perspective view of the spacer shim of FIG. 6;

FIG. 8 is a side view of the spacer shim of FIG. 6;

FIG. 9 is a perspective view of a tibial cutting template assembly according to the invention;

FIG. 10 is a top view of the tibial cutting template assembly of FIG. 9;

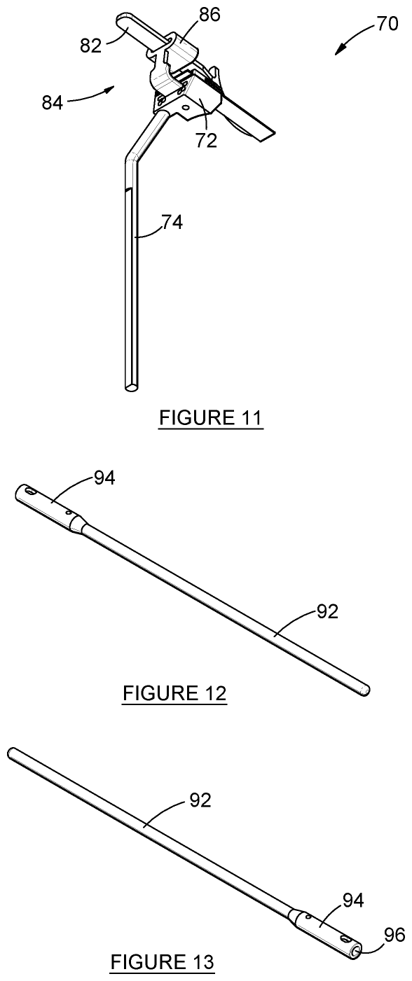

FIG. 11 is a bottom perspective view of the tibial cutting template assembly of FIG. 9;

FIG. 12 is a first perspective view of an intramedullary rod according to the invention;

FIG. 13 is a second perspective view of the intramedullary rod of FIG. 12, showing detail of a trailing end thereof;

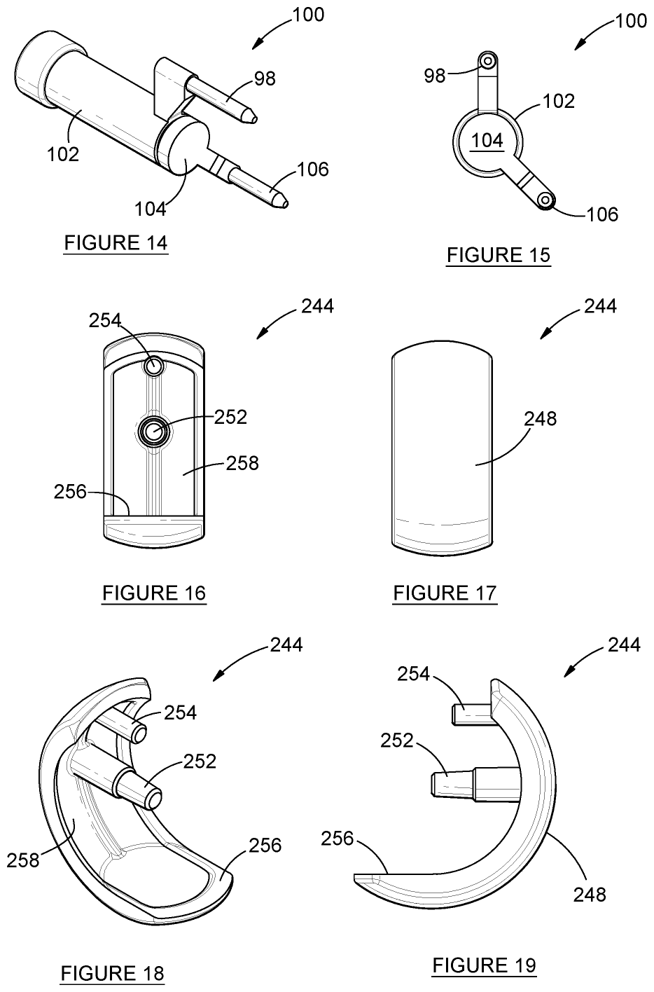

FIG. 14 is a perspective view of a connector device for connecting the intramedullary rod of FIG. 12 and the femoral cutting template of FIG. 1;

FIG. 15 is a front view of the connector device of FIG. 14;

FIG. 16 is a front view of a femoral component used during a UKA in accordance with the invention, the front view showing detail of inner, bone-connecting parts of the femoral component;

FIG. 17 is a rear view of the femoral component of FIG. 16, showing an outer bearing surface thereof;

FIG. 18 is a perspective view of the femoral component of FIG. 16;

FIG. 19 is a side view of the femoral component of FIG. 16;

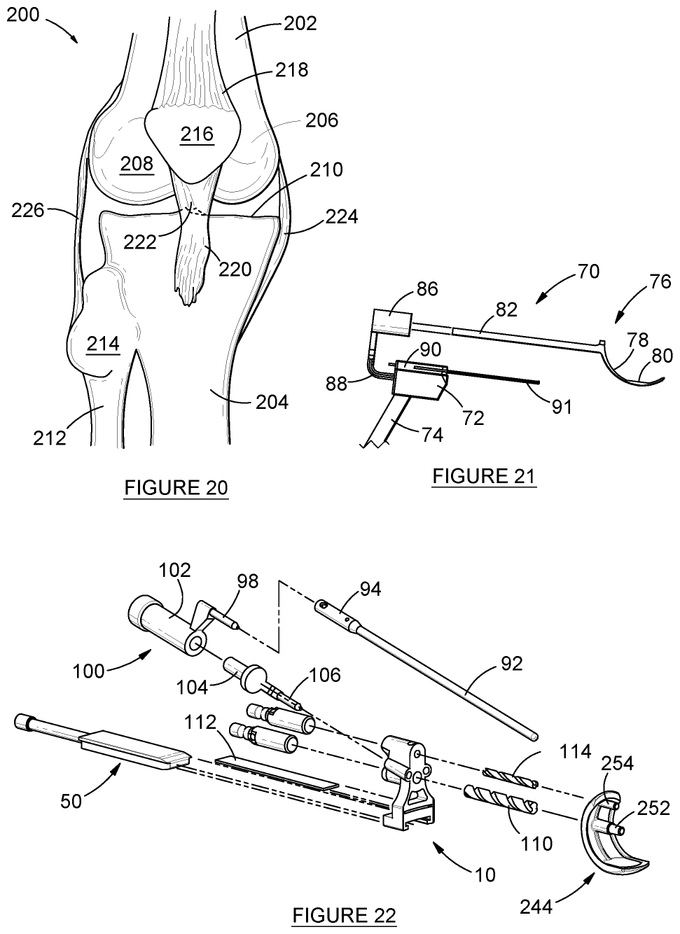

FIG. 20 is an anterior view of a normal human knee, showing the major bones, tendons and ligaments constituting a human knee joint;

FIG. 21 is an exploded side view of the tibial cutting template assembly of FIG. 9;

FIG. 22 is an exploded view of an assembly comprising the femoral cutting template of FIG. 1, the spacer shim of FIG. 6, the intramedullary rod of FIG. 12, the connector device of FIG. 14, and the femoral component of FIG. 16;

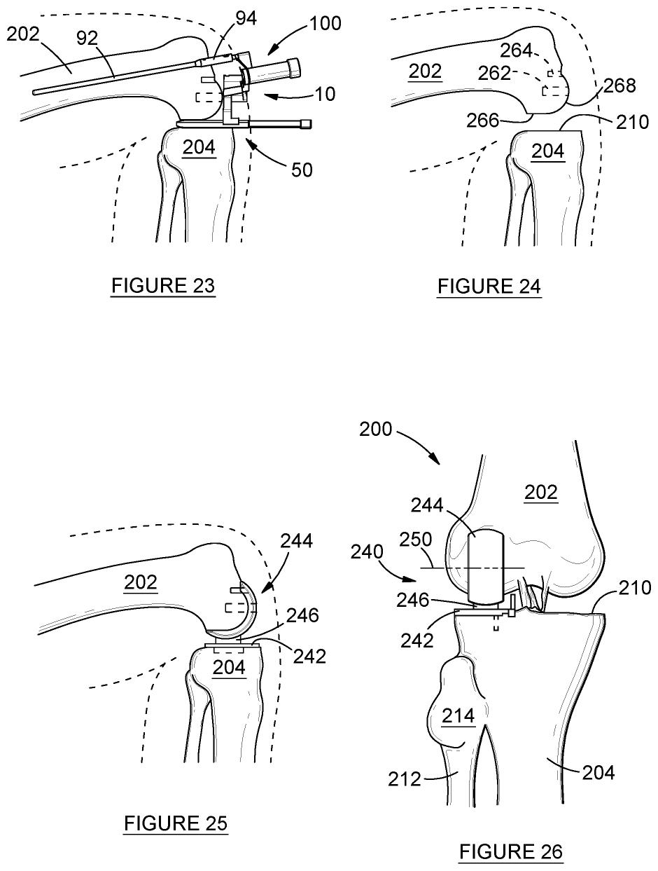

FIG. 23 is a lateral view of the human knee of FIG. 20, during a lateral unicondylar knee arthroplasty (UKA), with the femoral cutting template of FIG. 1, the spacer shim of FIG. 6, the intramedullary rod of FIG. 12 and the connector device of FIG. 14 in situ;

FIG. 24 is a lateral view of the human knee of FIG. 20, after the bony tissue has been suitably prepared for the placement of the femoral component of FIG. 16 and a tibial component;

FIG. 25 is a lateral view of the human knee of FIG. 24, with the femoral component of FIG. 16 in situ, a tibial component and a spacer bearing (meniscal component); and

FIG. 26 is an anterior view of the human knee of FIG. 25, at the conclusion of the UKA, with the femoral component of FIG. 16, a tibial component and a spacer bearing (meniscal component) in situ.

### DESCRIPTION OF A PREFERRED EMBODIMENT OF THE INVENTION

For the purpose of further background and illustration, FIG. 20 shows an anterior view of the major bones, ligaments and tendons constituting a typical human knee joint 200. The knee joint 200 joins the femur 202 to the tibia 204, allowing articulation between the femur 202 and the tibia 204 from a flexed to an extended condition. Distally, the femur 202 terminates in a medial femoral condyle 206 and a lateral femoral condyle 208. Proximally, the tibia terminates in a tibial plateau 210. Additional to the tibia 204, the fibula 212 also extends between the knee and the ankle (not shown). The superior aspect of the fibula 212 comprises a head 214, which is fixed to a lateral aspect of the tibia 204. The patella 216 is located anteriorly, between medial and lateral femoral condyles (206, 208). The patella 216 is fixed to the quadriceps muscle (not shown) via a quadriceps tendon 218 and to the tibia via a patellar tendon 220. The anterior cruciate ligament (ACL) 222 extends between the femur 202 and tibia 204. The tibial collateral ligament 224 extends between the femur 202 and tibia 204 on a medial aspect of the knee 200, while the fibular collateral ligament 226 extends between the femur 202 and tibia 204 on a lateral aspect of the knee 200. The ligaments and tendons of the knee 200 are jointly referred to as “stability providing structures”.

FIG. 26 shows an anterior view of the knee 200 after a lateral unicondylar knee prosthesis 240 has been implanted during a UKA. The patella 216 and stability providing structures are not shown, even though it will be appreciated that these remain in situ during and after the UKA. It will furthermore be appreciated that a medial unicondylar knee prosthesis (not shown) will have similar components, comprising substantially mirror-images of the components of the lateral unicondylar knee prosthesis 240. The lateral unicondylar knee prosthesis 240 comprises a tibial component 242, a femoral component 244 and a meniscal component or bearing 246 (which is also referred to as a “spacer bearing”). The spacer bearing 246 may be either fixed to the tibial component 242 (in which case the spacer bearing 246 is termed a “fixed spacer bearing”) or may be allowed two-dimensional sliding movement relative to the tibial component 242 (in which case the spacer bearing 246 is termed a “mobile spacer bearing”).

FIGS. 16 to 19 show a typical femoral component 244. The femoral component 244 comprises a highly polished outer femoral articular bearing surface 248. The bearing surface 248 is formed by an elliptical body which is substantially c-shaped and uni-radial about a first imaginary axis 250 which spans substantially in a medial-lateral direction of the knee, as shown in FIG. 26. The elliptical body is best illustrated in FIG. 19. The uni-radial elliptical body facilitates pivotal articular movement of the femoral component 244 about the first imaginary axis 250 and relative to the tibial component 242.

The femoral component 244 typically comprises first and second pegs (252, 254) (although it will be appreciated that a femoral component without a second peg 254 is feasible) and a flat surface 256. The first and second pins (252, 254) project from an inner surface or aspect 258 of the femoral component 244 and are received within prepared first and second holes (262, 264 on FIG. 24) within the femoral condyle (206, 208 as the case may be) in use.

The first and second pegs (252, 254) and the flat surface 256 extend substantially parallel to each other (as best illustrated in FIG. 19), to facilitate the placement of the femoral component 244. The first and second pegs (252, 254) and the flat surface 256 ensures that the femoral component 244 is properly fixed to the femur 202 and prevents relative movement (including rotational or pivotal relative movement) of the femoral component 244 relative to the femur 202.

Before placement of the femoral component 244 in situ, the femoral condyle needs to be resected and prepared. This is done by making use of a suitable femoral cutting template or guide.

A femoral cutting template or guide according to an example of the current invention is generally indicated by reference numeral 10 in the figures.

The femoral cutting template 10 comprises an attachment means 12 for releasably attaching the femoral cutting template 10 to a tibiofemoral spacer shim (which shim is indicated by reference numeral 50 in FIGS. 6 to 8). In use, and as is more fully described below, the spacer shim 50 is provided between the proximal tibia (tibial plateau 210 which may typically have been resected for the purpose of receiving the tibial component as aforementioned) and a surface of one of the medial and lateral femoral condyles (206, 208) of a patient (not shown).

The femoral cutting template 10 furthermore comprises a main body 14 defining a first guide aperture which is used to guide a first tool (such as first drill bit 110), while creating a first receiving formation in the femoral condyle (the relevant one of 206 and 208). The first receiving formation receives a first part of the femoral component in use.

The main body 14 also defines a second guide aperture which is used to guide a second tool (such as sideways oscillating cutting blade 112), while creating a second receiving formation in or on the femoral condyle (the relevant one of 206 and 208). The second receiving formation receives a second part of the femoral component in use.

According to the example embodiment shown in the figures, the first guide aperture typically takes the form of a first guide hole 16 and the second guide aperture typically takes the form of guide slot 18. The first receiving formation takes the form of a first femoral hole 262 which is drilled into the femoral condyle (the relevant one of 206 and 208) by the first drill bit 110. The sideways oscillating cutting blade 112 is used for resecting a portion of the femoral condyle (the relevant one of 206 and 208) (the resected portion of the femoral condyle constituting the second receiving formation, shown as surface 266).

The first femoral hole 262 is provided for receiving the first part of the femoral component 244, which takes the form of the first peg 252, while the resected portion of the condyle is provided for receiving the second part of the femoral component 244, namely the flat surface 256.

As is best illustrated in FIG. 3, the guide slot 18 is spaced a first predetermined distance 20 from the first guide hole 16, and a second predetermined distance 22 from the attachment means 12. The first and second distances (20, 22) are determined by a specific geometry of the femoral component 244.

The main body 14 further defines a third guide aperture in the form of a second guide hole 24. As is described in more detail below, the second guide hole 24 is used to guide a second drill bit 114 when drilling a second femoral hole 264 into the femoral condyle (the relevant one of 206 and 208). The second femoral hole 264 is provided for receiving the second peg 254 of the femoral component 244.

The first and second guide holes (16, 24) are spaced a third predetermined distance 26 from each other, in accordance with the geometry of a specific femoral component 244 and are arranged substantially along a centre of the main body 14. The first and second guide holes (16, 24) are provided substantially parallel to each other, as the first and second pegs (252, 254) are also substantially parallel. The first and second guide holes (16, 24) therefore facilitate the placement of the femoral component 244.

The main body 14 furthermore defines a viewing aperture 28 between the guide slot 18 and the first guide hole 16. In use, when the femoral cutting template 10 is in situ and used to resect the portion of the femoral condyle, the viewing aperture 28 facilitates substantially uninhibited viewing access to an anterior aspect of the knee joint 200, from which the portion of the femoral condyle is resected. This enables the surgeon to more accurately exercise control over the resection of the portion of the femoral condyle.

The main body 14 further defines a first securing formation, in the form of a first securing hole 30 and a second securing formation, in the form of a second securing hole 32. The first securing hole 30 is provided towards a first, left portion of the main body 14 and is arranged obliquely relative to the first and second guide holes (16, 24). From a front face of the template, the first securing hole 30 extends downwardly and outwardly towards the left side of the main body 14.

The second securing hole 32 is provided towards a second, right portion of the main body 14 and is arranged obliquely relative to the first and second guide holes (16, 24). From the front face of the template 10, the second securing hole 32 extends downwardly and outwardly towards the right side of the main body 14.

As is described in more detail below, the first and second securing holes (30, 32) may be used as points of fixation, to facilitate the proper alignment of the femoral cutting template 10 relative to the medial or lateral condyle (206, 208) as the case may be, during a medial UKA or lateral UKA respectively. The first and second securing holes (30, 32) are provided to receive, in use, a first pin 98 of a connector device 100 (which is shown in FIGS. 14 and 15) which is releasably fixed to an intramedullary rod 92 as discussed in more detail below.

The attachment means 12 comprises a first hook shaped formation 34 and second hook-shaped formation 36, to form a pair projecting from the main body 14. The first and second hook-shaped formations (34, 36) are provided in an opposing arrangement to define a T-shaped slot therebetween. In use, the attachment means 12 slides over the spacer shim 50, with the first and second hook-shaped formations (34, 36) receiving a rim formation 62 and 64 of the spacer shim 50, thereby releasably to attach the template to the spacer shim. Since the first, second and third predetermined distances (20, 22 and 26) are determined relative to the attachment means 12, the spacing of the first and second holes (262, 264) and the resection of the femoral condyle is accurate and controlled.

The femoral cutting template 10 is typically manufactured from titanium and may be of unitary construction. The femoral cutting template 10 may be machined or cast. Alternatively, the femoral cutting template may be manufactured from a suitable metal such as aluminium or stainless steel.

A face 40 of the of the main body 14 which is operatively arranged towards the anterior aspect of the femur, is concave, to facilitate placement of the femoral cutting template 10 in close proximity to the femoral condyle (206 or 208).

The spacer shim 50 comprises a first surface 52 (which operatively constitutes a bottom surface) which is in use positioned in contact with the tibial plateau 210. As will be described in more detail below, the tibial plateau is resected before placement of the spacer shim 50. The spacer shim 50 furthermore comprises a second surface 54 (which operatively constitutes a top surface) which is, in use, positioned in contact with a surface of either the medial or lateral condyle (206, 208) respectively, depending on whether a medial or lateral UKA is performed. As described in more detail below, during the placement of spacer shim 50, the femoral condyle is not yet resected.

The first and second surfaces (52, 54) are separated by a spacing distance 56 (defined by the thickness of the spacer shim 50). The first and second surfaces (52, 54) extend substantially parallel to each other, to enable the placement of the spacer shim 50 into the tibiofemoral compartment by advancing a front end 58 of the spacer shim 50 into the tibiofemoral compartment from an anterior portion of the knee, in a posterior direction. In this way, and as described in more detail below, the spacer shim 50 may be advanced into the tibiofemoral compartment of the knee 200 without having to resect or disturb any of the stability-providing structures of the knee. A front end-portion of the spacer shim 50 may furthermore be chamfered to facilitate the easy insertion thereof between the resected tibial plateau 210 and the femoral condyle (206, 208).

The spacer shim 50 furthermore comprises an attachment formation which is used to attach the femoral cutting template 10 to the spacer shim 50 in releasable fashion.

The attachment formation is in the form of the first and second longitudinally extending lateral rims (62, 64) projecting towards the sides of the spacer shim 50. The first and second rims (62, 64) have top surfaces which are substantially flush with the top surface 54. When viewed from the front, the spacer shim 50 is substantially T-shaped and shaped to fit into the T-shaped slot 38 of the femoral cutting template 10. A tolerance between the T-shaped slot 38 and the spacer shim 50 may be loose enough to allow the spacer shim 50 and femoral cutting template 10 to slide freely relative to one another, yet fine enough to prevent large or significant sideways displacement or pivotal movement. The fit between the T-shaped slot 38 and the spacer shim 50 is therefore closer than a rattle fit.

The spacer shim 50 is provided with a handle 66 to facilitate insertion and removal of the spacer shim 50 into and from the tibiofemoral compartment. The handle 66 is removable to enable easy packaging and transport. A front portion of the handle (not shown) is threaded, to be received within a tapped blind hole provided on a rear surface of the spacer shim 50.

The use and interaction of the different components as described above will now be discussed in more detail with reference to a lateral UKA.

Tibiofemoral degeneration is diagnosed by known methods (for example, such as described in US Patent Application 20170231552A1, filed in the name of the current inventor). As a prerequisite for the performing of a UKA, the stability-providing structures need to be in place and provide adequate amounts of stability to the knee joint. In the case of severe damage to the stability providing structures, a total knee arthroplasty (TKA) instead of a UKA will be indicated.

During the surgical procedure (UKA), an anterior incision is made in the skin of the patient, to reveal the lateral tibiofemoral compartment.

A tibial cutting template which is indicated by reference numeral 70 in FIG. 9, comprises a guide body 72 and an alignment stem 74. The alignment stem 74 is placed substantially parallel to the tibia, and fastened in position towards the ankle, with a strap (not shown).

A femoral sizing device or spoon 76 is inserted between the tibial plateau 210 and the lateral femoral condyle 208. The femoral sizing spoon comprises an arched member 78. A number of different femoral sizing spoons 76 having different arch sizes are provided in a kit utilised during the UKA. The most suitable size femoral sizing spoon 76 is determined by the fit between the arched member 78 and the condyle 208. The arched member 78 comprises ridges 80 that cut into the bony tissue of the femoral condyle 208, to prevent sideways rotational movement of the femoral sizing spoon 76 relative to the condyle 208.

A suitably sized femoral component 244 is selected based on the size of the femoral sizing spoon 76. A handle 82 of the femoral sizing spoon 76 serves as a reference point to fix the height of the tibial cutting template 70 relative to the femur. Once the suitably sized femoral sizing spoon 76 is selected and positioned between the tibial plateau 210 and the femoral condyle 208, a connector device 84 comprising a sleeve 86 and an L-shaped spacing member 88 is attached to the femoral sizing spoon 76 by sliding the sleeve 86 over the handle 82. The L-shaped spacing member 88 is received within a suitable slot in the guide body 72. A blade guide 90 is provided on the guide body 72, to guide a blade used to resect the tibial plateau 210. The blade guide 90 is releasably fixable relative to the guide body 72. Different blade guides 90 having different thicknesses may be provided, so that the position of the blade 91 may be adjusted relative to the tibial plateau. By adjusting the position of the blade 91 relative to the tibial plateau 210, the thickness of the portion of the tibial plateau that will be resected is adjusted.

The specific geometry of the components that make up the tibial cutting template 70 results in a predetermined spacing between the tibial plateau 210 once resected and the bottom-most portion of the femoral condyle 208. The predetermined spacing may typically be in the order of 7 mm. In some cases, the amount of degradation, elasticity and/or natural length of the stability providing structures may cause the predetermined spacing to exceed 7 mm. In such a case, a spacer shim having a larger spacing distance 56 is provided. For this purpose, spacer shims 50 having spacing distances 56 of 8 mm, 9 mm and 10 mm respectively may be provided. A suitable spacer shim 50 is then used to elevate the femoral condyle in balance with the natural length of the ligaments to ensure anatomical and central orientation and rotation of the femoral component once implanted.

By fastening the alignment stem 74 to the lower limb of the patient, proper alignment of the guide body 72 is ensured, whilst fixing the height of the blade guide 90 by utilising the handle 82 of the femoral sizing spoon 76 as a reference height, the height of the blade guide 90 relative to the femoral condyle 208, as well as the tibial plateau 210 is ensured. Once the alignment and placement of the blade guide 90 is complete, the guide body may be fastened to the tibia 204 by driving a nail through a suitable hole, and into the tibia 204.

A blade 91, which oscillates sideways, is projected through a suitable slot within the blade guide 90, and a suitable portion of the tibial plateau is resected and removed. The tibial plateau is resected to remove portions of the bony material of the tibial plateau that may be damaged, to create space for the tibial component 242 within the tibiofemoral compartment and to create a flat surface for the tibial component 242 to sit, or be supported, on. The slot within the blade guide 90 ensures that the blade 91 is angled relative to the tibial plateau 210 correctly.

The resected tibial plateau now serves as a new reference point for resection of the femoral condyle 208.

A hole is created between the medial and lateral condyles (206, 208) and the intramedullary rod 92 is advanced therethrough and into the femur 202. The exact positioning of the hole is determined by the surgeon based on expertise. A trailing end portion of the of the intramedullary rod 92 comprises a cylindrical body 94 defining a receiving aperture 96 for receiving a first pin 98 of a connector device 100. The receiving aperture 96 is concentric with the intramedullary rod 92, thereby to fix the alignment of connector device 100 relative to the femur 202.

The connector device comprises a main body 102 defining a central cylindrical hole for receiving an inner body. The first pin 98 is spaced from, and extends from, the main body 102. A second pin 106 is spaced from, and extends from, an inner body 104 of the connector device 100. The main—and inner bodies (102, 104) are free to pivot relative to each other, so that the distance between the first and second pins (98, 106) may effectively be changed. The alignment of the first and second pins (98, 106) however stays fixed relative to the intramedullary rod and therefore the femur 202.

After the resection of the tibial plateau, the spacer shim 50 is advanced into the tibiofemoral compartment, and therefore between the resected tibial plateau and the femoral condyle 208. The first surface 52 therefore abuts the resected tibial plateau. Different spacer shims 50, having different spacing distances 56 as mentioned above are provided in the kit. A suitable spacer shim 50 is identified when the femoral condyle is elevated such that a suitable amount of pressure is exerted between the resected tibial plateau and the femoral condyle 208. The amount of degradation, elasticity and/or natural length of the stability providing structures will influence the suitable spacing distance 56.

Once the correct spacer shim 50 is securely positioned within the tibiofemoral compartment (and held in place by the pressure exerted thereon by the tibial plateau 210 and the lateral femoral condyle 208), the femoral cutting template 10 is releasably attached to the spacer shim 50, by sliding the T-shaped slot 38 over the first and second rims (62, 64) of the spacer shim 50. The femoral cutting template 10 is positioned so that the face 40 is in close proximity to the anterior aspect of the femoral condyle 208. The curvature of the face 40 enables the femoral cutting template 10 to be positioned in close proximity to the femoral condyle 208.

The first pin 98 of the connector device 100 is inserted into the receiving aperture 96 of the intramedullary rod 92, and the second pin 106 of the connector device 100 is inserted into the second securing hole 32 of the femoral cutting template 10. The femoral cutting template 10 is now properly positioned in place. The height, orientation and horizontal position of the femoral cutting template 10 is determined by the spacer shim 50 and the connector device 100.

The first drill bit 110 is advanced through the first guide hole 16 and the first hole 262 is drilled into the femoral condyle 208. The second drill bit 114 is advanced through the second guide hole 24 and the second hole 264 is drilled into the femoral condyle 208. In each instance the drill (not shown) is detached from the first and second drill bits (110, 114) leaving the first and second drill bits (110, 114) in situ. The first and second drill bits (110, 14) therefore act as further points of fixation and alignment of the femoral cutting template 10 relative to the femoral condyle 208.

A sideways oscillating saw blade 112 is advanced through the guide slot 18 and a portion of the femoral condyle 208 is resected and removed. The resection of the femoral condyle provides a surface 266 for the flat surface 256 of the femoral component 244 to sit on. The surface 266 is substantially parallel to the first hole 262 so that the femoral component 244 may easily be advanced into position from an anterior aspect of the tibiofemoral compartment in a sliding fashion. The viewing aperture 28 provides the surgeon uninhibited view of the femoral condyle 208 while resecting the portion of the femoral condyle 208. Since the first guide hole 16 and the guide slot 18 are both provided on the femoral cutting template 10, the spacing between the resected tibial plateau 210, the resected femoral condyle 208 and the first guide hole 262 is fixed. This results in a more accurate preparation of the bony fixation points, and in turn, more accurate placement of the tibial and femoral components (242, 244). Accurate placement of the tibial and femoral components (242, 244) is essential for the success of the arthroplasty.

It will be appreciated that the portion of the femoral condyle 208 that is resected, is a posterior portion of the lateral condyle 208. In order to access this portion of the condyle 208 from an anterior aspect of the knee, the knee is held in flexion during the resection of the condyle 208.

Next, the femoral condyle is reamed to provide a substantially spherical surface 268 for the inner surface 258 of the femoral component 244 to sit on in use.

Preparation of the bony tissue has now been completed. The femoral cutting template 10 is removed, and the tibial and femoral components (242, 244), and the spacer bearing 246 is inserted according to known methods. The thickness of the spacer bearing is determined by the size of the spacer shim 50 that was used.

By fixing the position of the femoral cutting guide 10 relative to the resected tibial plateau 210, and to the femur 202, and since the first guide hole 16 and the guide slot 18 are provided within a single body 14, the accuracy of the first hole 262 and the resected surface 266 of the femoral condyle is ensured. The aforementioned provides the surgeon performing the UKA with more confidence, while ensuring improved central placement and more repeatable outcomes. This encourages the use of UKAs over TKAs in suitable cases.

The viewing aperture provides the surgeon with more control over the precise placement of the femoral component and resection of the femoral condyle, again ensuring more accurate and repeatable results. The precise placement of the femoral component is required to ensure a proper articulation of the femoral component, and a suitable rotational or pivotal angle of the femoral component.

It will be appreciated that the first and second securing holes are substantially mirror images of each other, and that only one will be used during a specific UKA. The side in which the UKA is performed (such as medial or lateral) will determine which of the first and second securing holes will be used. In this way, a single template 10 is suitable for use during both lateral and medial UKAs.

It will be appreciated by those skilled in the art that the invention is not limited to the precise details as described herein and that many variations are possible without departing from the scope and spirit of the current disclosure. For example, the spacings 20, 22, 26 of the femoral cutting template 10 are specific to the type of femoral component 244 utilised. The layout and geometry of the femoral component 244 may differ from the current disclosure. For example, a femoral component without a second peg 254 may be provided, in which case the second guide hole 24 may be omitted. Also, the femoral component may comprise further bone attachment formations. Further guide holes and slots may therefore be provided through the femoral cutting template 10 to accommodate the drilling into and resection of the femoral condyle, to provide holes and slots for receiving such additional bone attachment formations. It will be appreciated that the second guide aperture 18 may in an alternative embodiment (which is not shown), comprise a secondary guide hole (not shown), in which case the second tool will be a secondary drill bit (not shown) used to create a secondary hole in the femoral condyle, for receiving a corresponding secondary peg (not shown) of the femoral component.

It will further be appreciated that the foregoing examples have been provided merely for the purpose of explanation and are in no way to be construed as limiting of the present invention.

It will be understood that references to a tibial plateau, may include references to a resected portion of a tibial plateau.

1. A femoral cutting template comprising:
attachment means for releasably attaching the template to a tibiofemoral spacer shim which is operatively provided between a proximal tibia and a femoral condyle;
a main body defining a first guide aperture for guiding a first tool, used for creating a first receiving formation in the femoral condyle, the first receiving formation for receiving a first part of a femoral component; and
a second guide aperture defined within the main body, for in use, guiding a second tool used to create a second receiving formation in the femoral condyle, the second receiving formation for receiving a second part of the femoral component, the second guide aperture spaced a first predetermined distance from the first guide aperture, and a second predetermined distance from the attachment means.

2. A femoral cutting template according to claim 1, wherein the first guide aperture comprises a first guide hole, the first tool comprises a first drill bit and the first part of the femoral component comprises a first peg located on an inner aspect of the femoral component.

3. A femoral cutting template according to claim 2, wherein the second guide aperture comprises a guide slot, the second tool comprises a cutting blade and the second part of the femoral component comprises a flat surface on an inner aspect of the femoral component.

4. A femoral cutting template according to claim 3, wherein the main body defines a third guide aperture in the form of a second guide hole, for guiding a second drill bit, used for creating a second hole in the femoral condyle, the second hole for receiving a second peg located on an inner aspect of the femoral component.

5. (canceled)

6. A femoral cutting template according to claim 4, wherein the main body defines a viewing aperture between the first and second guide apertures, for operatively providing viewing access of an anterior aspect of the femoral condyle.

7. A femoral cutting template according to claim 6, wherein the main body defines a first securing formation, in the form of a first securing hole, arranged towards a first side portion of the main body, such that the first securing hole extends obliquely relative to the first guide aperture.

8. A femoral cutting template according to claim 7, wherein the first side portion is a left side portion of the main body, and wherein the first securing hole is provided for releasably receiving a pin of a connector device which, in use, is fixed to an intramedullary rod during a medial unicondylar arthroplasty.

9. A femoral cutting template according to claim 8, wherein the first securing hole extends substantially downwardly and outwardly from an anterior face of the main body, towards a posterior face of the main body.

10. A femoral cutting template according to claim 9, wherein the main body defines a second securing formation in the form of a second securing hole, arranged towards a second side portion of the main body, such that the second securing hole extends obliquely relative to the first guide aperture.

11. A femoral cutting template according to claim 10, wherein the second side portion is a right side portion of the main body, and wherein the second securing hole is provided for releasably receiving a pin of a connector device which, in use, is releasably fixed to an intramedullary rod during a lateral unicondylar arthroplasty.

12. A femoral cutting template according to claim 11, wherein the second securing hole extends substantially downwardly and outwardly from an anterior face of the main body, towards a posterior face of the main body.

13. A femoral cutting template according to claim 12, wherein the attachment means comprises first and second opposing hook-shaped formations projecting from the main body, the first and second hook-shaped formations constituting a pair defining a T-shaped slot therebetween, for in use, receiving rim formations formed on the spacer shim, thereby releasably to attach the femoral cutting template to the spacer shim.

14. A femoral cutting template according to claim 13, wherein a posterior face of the of the main body, which is operatively arranged to face the femoral condyle, is concave.

15. A femoral cutting template according to claim 14, which is of unitary construction and manufactured from a metal selected from the group comprising titanium, aluminium and ferrous alloys including stainless steel.

16.-17. (canceled)

18. A femoral cutting template according to claim 2, wherein the second guide aperture comprises a secondary guide hole, the second tool comprises a secondary drill bit and the second formation comprises a secondary peg located on an inner aspect of the femoral component.

19. A tibiofemoral spacer shim comprising:
a first surface for operatively contacting a proximal tibia;
a second surface for operatively contacting a surface of a femoral condyle, the first and second surfaces separated by a spacing distance; and
an attachment formation, comprising first and second, longitudinally extending lateral shoulder formations, for facilitating a femoral cutting template to be releasably attached thereto.

20. (canceled)

21. A tibiofemoral spacer shim according to claim 19, wherein the first and second lateral shoulder formations have upper surfaces which are substantially flush with the second surface, such that the shim is in the form of a substantially T-shaped elongate body.

22. A tibiofemoral spacer shim according to claim 21, further comprising a removable handle, in use extending rearwardly from a rear portion of the elongate body.

23. (canceled)

24. A femoral cutting assembly, consisting of:
a tibiofemoral spacer shim, comprising first and second surfaces separated by a spacing distance, the first and second surfaces for operatively contacting a proximal tibia and a surface of a femoral condyle respectively; and
a femoral cutting template, releasably attached via an attachment means to the tibiofemoral spacer shim, the femoral cutting template comprising:
a main body defining a first guide aperture for guiding a first tool, used for creating a first receiving formation in the femoral condyle, the first receiving formation for receiving a first part of a femoral component; and
a second guide aperture defined within the main body for guiding a second tool used to create a second receiving formation in the femoral condyle, the second receiving formation for receiving a second part of the femoral component, the second guide aperture spaced a first predetermined distance from the first guide aperture, and a second predetermined distance from the attachment means.

25. A method of preparing a femoral condyle for the placement of a femoral component forming part of a unicondylar knee prosthesis, the method comprising the steps of:
placing a tibiofemoral spacing shim between a proximal tibia and a surface of the femoral condyle;
releasably attaching a femoral cutting template via an attaching means to the tibiofemoral spacing shim, the femoral cutting template comprising first and second guide apertures;
providing a first tool into the first guide aperture and utilising the first tool to create a first receiving formation in the femoral condyle; and
providing a second tool into the second guide aperture and utilising the second tool to create a second receiving formation in the femoral condyle.

26.-29. (canceled)
